(19) United States
(12) Patent Application Publication
(10) Pub. No.: US 2011/0060013 A1
(43) Pub. Date: Mar. 10, 2011

(54) THIAZOLES AND PYRAZOLES USEFUL AS KINASE INHIBITORS

(75) Inventors: Michael Paul Mortimore, Burford (GB); Christopher John Davis, Salisbury (GB); Julian M.C. Golec, Woolstone (GB); John Studley, Abingdon (GB); Daniel David Robinson, Abingdon (GB)

(73) Assignee: VERTEX PHARMACEUTICALS INCORPORATED, Cambridge, MA (US)

(21) Appl. No.: 12/601,026

(22) Filed: Nov. 20, 2009

Related U.S. Application Data
(63) Continuation of application No. PCT/US2008/062331, filed on May 24, 2008.
(60) Provisional application No. 60/939,876, filed on May 24, 2007.

(51) Int. Cl. A61K 31/4439; C07D 401/14; C07D 417/14; A61P 35/00

(52) U.S. Cl. 514/341; 546/275.4; 546/270.7; 514/342

(57) ABSTRACT

The present invention relates to compounds useful as inhibitors of Aurora protein kinases. The invention also provides pharmaceutically acceptable compositions comprising those compounds and methods of using the compounds and compositions in the treatment of various disease, conditions, and disorders. The invention also provides processes for preparing compounds of the invention.

### TECHNICAL FIELD OF THE INVENTION

This application is a continuation application of International Patent Application No. PCT/US2008/062331, filed on May 24, 2008, which in turn claims the benefit under 35 U.S.C. §119, of U.S. Provisional patent application No. 60/939,876, filed May 24, 2007, entitled “THIAZOLES AND PYRAZOLES USEFUL AS KINASE INHIBITORS”, and the entire contents of these applications are hereby incorporated by reference

The present invention relates to compounds useful as inhibitors of Aurora protein kinases. The invention also relates to pharmaceutically acceptable compositions comprising the compounds of the invention, methods of using the compounds and compositions in the treatment of various disorders, and processes for preparing the compounds.

### BACKGROUND OF THE INVENTION

The Aurora proteins are a family of three related serine/threonine kinases (termed Aurora-A, -B and -C) that are essential for progression through the mitotic phase of cell cycle. Specifically Aurora-A plays a crucial role in centrosome maturation and segregation, formation of the mitotic spindle and faithful segregation of chromosomes. Aurora-B is a chromosomal passenger protein that plays a central role in regulating the alignment of chromosomes on the meta-phase plate, the spindle assembly checkpoint and for the correct completion of cytokinesis.

Overexpression of Aurora-A, -B or -C has been observed in a range of human cancers including colorectal, ovarian, gastric and invasive duct adenocarcinomas.

A number of studies have now demonstrated that depletion or inhibition of Aurora-A or -B in human cancer cell lines by siRNA, dominant negative antibodies or neutralizing antibodies disrupts progression through mitosis with accumulation of cells with 4N DNA, and in some cases this is followed by endoreduplication and cell death.

The Aurora kinases are attractive targets due to their association with numerous human cancers and the roles they play in the proliferation of these cancer cells. Accordingly, there is a need for compounds that inhibit Aurora kinases.

### SUMMARY OF THE INVENTION

This invention provides compounds and pharmaceutically acceptable compositions thereof that are useful as inhibitors of Aurora protein kinases. These compounds are represented by formula I:

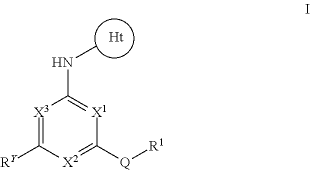

or a pharmaceutically acceptable salt thereof, wherein the variables are as defined herein.

These compounds and pharmaceutically acceptable compositions thereof are useful for inhibiting kinases in vitro, in vivo, and ex vivo. Such uses include treating or preventing myeloproliferative disorders and proliferative disorders such as melanoma, myeloma, leukemia, lymphoma, neuroblastoma, and cancer. Other uses include the study of kinases in biological and pathological phenomena; the study of intracellular signal transduction pathways mediated by such kinases; and the comparative evaluation of new kinase inhibitors.

### DETAILED DESCRIPTION OF THE INVENTION

One embodiment of this invention provides a compound of formula I:

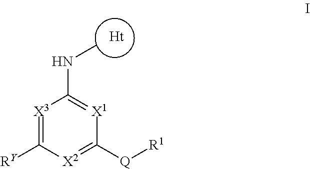

or a pharmaceutically acceptable salt thereof, wherein:
X¹ is N or CH;
X² is N or CH;
X³ is N or CR^{X};
provided that when X³ is CR^{X}, only one of X¹ and X² is N; and
provided that at least one of X¹, X² and X³ is N;
Ht is thiazole or pyrazole, wherein each ring is optionally and independently substituted with R² and R^{2′};
Q is —O—, —NR′—, —S—, —C(═O)—, or —C(R′)₂—;
R^{x} is H or F;
R^{Y} is —Z—R¹⁰;
R¹ is T-(Ring D);
Ring D is a 5-7 membered monocyclic aryl or heteroaryl ring, wherein said heteroaryl has 1-4 ring heteroatoms selected from O, N, or S; Ring D can optionally be fused with Ring D′;
Ring D′ is a 5-8 aromatic, partially saturated, or fully unsaturated ring containing 0-4 ring heteroatoms selected from nitrogen, oxygen, or sulfur;
Ring D and Ring D′ are each independently and optionally substituted with 0-4 occurrences of oxo or —W—R⁵;
each T is independently a C₁₋₄ alkylidene chain or is absent;
R² is H, C₁₋₃ alkyl, or cyclopropyl;
R^{2′} is H;
each Z and W is independently absent or a C₁₋₁₀ alkylidene chain wherein up to six methylene units of the alkylidene chain are optionally replaced by V;
each V is selected from —O—, —C(═O)—, —S(O)—, —S(O)₂—, —S—, or —N(R⁴)—;
each R⁵ is independently —R, -halo, —OR, —C(═O)R, —CO₂R, —COCOR, COCH₂COR, —NO₂, —CN, —S(O)R, —S(O)₂R, —SR, —N(R⁴)₂, —CON(R⁷)₂, —SO₂N(R⁷)₂, —OC(═O)R, —N(R⁷)COR, —N(R⁷)CO₂ (C₁₋₆ aliphatic), —N(R⁴)N(R⁴)₂, —C═NN(R⁴)₂, —C═N—OR, —N(R⁷)CON(R⁷)₂, —N(R⁷)SO₂N(R⁷)₂, —N(R⁴)SO₂R, or —OC(═O)N(R⁷)₂;
each R is H, a C₁₋₆ aliphatic group, a C₆₋₁₀ aryl ring, a heteroaryl ring having 5-10 ring atoms, or a heterocyclyl ring having 4-10 ring atoms; wherein said heteroaryl or heterocyclyl ring has 1-4 ring heteroatoms selected from nitrogen, oxygen, or sulfur; R is optionally substituted with 0-6 R⁹;
each R⁴ is —R⁷, —COR⁷, —CO₂R⁷, —CON(R⁷)₂, or —SO₂R⁷;
each R⁷ is independently H or C₁₋₆ aliphatic optionally substituted with 1-6 halo or —O(C₁₋₆alkyl); or two R⁷ on the same nitrogen are taken together with the nitrogen to form an optionally substituted 4-8 membered heterocyclyl or heteroaryl ring containing 1-4 heteroatoms selected from nitrogen, oxygen, or sulfur;
each R⁹ is —R′, -halo, —OR′, —C(═O)R′, —CO₂R′, —COCOR′, COCH₂COR′, —NO₂, —CN, —S(O)R′, —S(O)₂R′, —SR′, —N(R′)₂, —CON(R′)₂, —SO₂N(R′)₂, —OC(═O)R′, —N(R′)COR′, —N(R′)CO₂(C₁₋₆ aliphatic), —N(R′)N(R′)₂, —N(R′)CON(R′)₂, —N(R′)SO₂N(R′)₂, —N(R′)SO₂R′, —OC(═O)N(R′)₂, ═NN(R′)₂, ═N—OR′, or ═O;
each R¹⁰ is a 5-6 membered heterocyclic ring containing 1 heteroatom selected from O, N, or S; each R¹⁰ is optionally substituted with 0-6 occurrences of J;
each J is independently R, -halo, —OR, oxo, —C(═O)R, —CO₂R, —COCOR, —COCH₂COR, —NO₂, —CN, —S(O)R, —S(O)₂R, —SR, —N(R⁴)₂, —CON(R⁷)₂, —SO₂N(R⁷)2, —OC(═O)R, —N(R⁷)COR, —N(R⁷)CO₂(C₁₋₆ aliphatic), —N(R⁴)N(R⁴)₂, ═NN(R⁴)₂, ═N—OR, —N(R⁷)CON(R⁷)₂, —N(R⁷)SO₂N(R⁷)₂, —N(R⁴)SO₂R, —OC(═O)N(R⁷)₂, or —OP(═O)(OR″)₂; or
2 J groups, on the same atom or on different atoms, together with the atom(s) to which they are bound, form a 3-8 membered saturated, partially saturated, or unsaturated ring having 0-2 heteroatoms selected from O, N, or S; wherein 1-4 hydrogen atoms on the ring formed by the 2 J groups is optionally replaced with J^{R}; or two hydrogen atoms on the ring are optionally replaced with oxo or a spiro-attached C₃₋₄ cycloalkyl; wherein said C₃₋₄ alkyl is optionally substituted with 1-3 fluorine;
each J^{R} is F or R^{7′};
each R^{7′} is independently C₁₋₆ aliphatic; —O(C₁₋₆ aliphatic); or a 5-6 membered heteroaryl containing 1-4 heteroatoms selected from O, N, or S; each R^{7′} is optionally substituted with 0-3 J⁷;
J⁷ is independently NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂ (C₁₋₄aliphatic), O(haloC₁₋₄aliphatic), or haloC₁₋₄aliphatic;
each R′ is independently H or a C₁₋₆ aliphatic group; or two R′, together with atom(s) to which they are bound, form a 3-6 membered carbocyclyl or a 3-6 membered heterocyclyl containing 0-1 heteroatoms selected from O, N, or S; and
each R″ is independently H or C₁₋₂alkyl.

In some embodiments, X¹ is N. In other embodiments, X¹ is CH. In some embodiments, X² is N. In other embodiments, X² is CH. In some embodiments, X³ is CR^{X}. In other embodiments, X³ is N. In some embodiments, X¹, X², and X³ are all N. In other embodiments, X¹ is N, X² is CH, and X³ is CR^{X}. In yet other embodiments, X¹ is CH, X² is N, and X³ is CR^{X}. In some embodiments, X¹ is N, X² is CH, and X³ is N. In other embodiments, X¹ is CH, X² is CH, and X³ is N.

Some embodiments provide compounds of formulae I-a to I-f, wherein the variables are as defined herein.

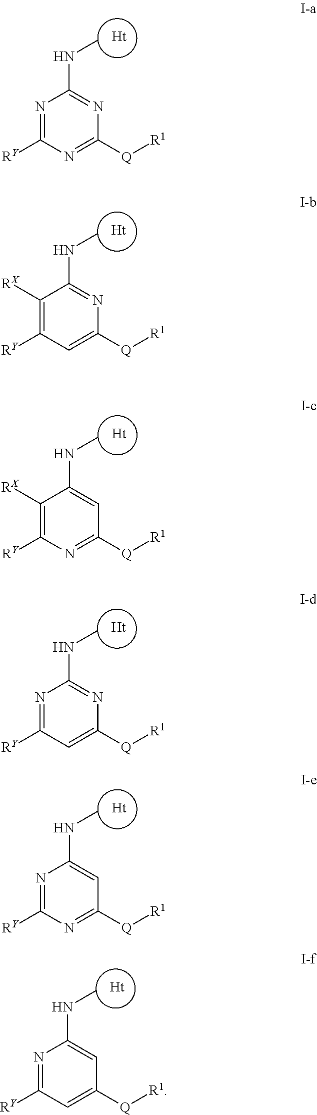

In one aspect of the invention, Ht is

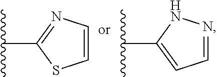

wherein each ring is optionally and independently substituted with R² and R^{2′}. In some embodiments, Ht is

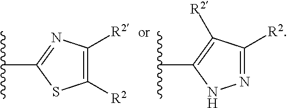

In some embodiments, Q is —S—. In other embodiments, Q is —O—. In yet other embodiments, Q is —C(═O)—. In some embodiments, Q is —C(R′)₂—.

In some embodiments, R² is H or C₁₋₃ alkyl.

In another embodiment, Ring D is a 5-6 membered monocyclic aryl or heteroaryl ring. In some embodiments, Ring D is a 6-membered monocyclic aryl or heteroaryl ring. In some embodiments, Ring D is fused with Ring D′.

In one aspect of the invention, Ring D-D′ is an 8-12 membered bicyclic aryl or heteroaryl containing 1-5 heteroatoms selected from nitrogen, oxygen, or sulfur. In some embodiments, Ring D-D′ is a 6:6 ring system. In some embodiments, Ring D-D′ is quinoline. In other embodiments, Ring D-D′ is a 6:5 ring system. In some embodiments, said 6:5 ring system contains 2 nitrogen atoms. In some embodiments, Ring D-D′ is a benzimidazole, indazole, or imidazopyridine ring. In other embodiments, Ring D-D′ is a benzimidazole ring. In another aspect of the invention, Ring D is a 5-6 membered monocyclic aryl or heteroaryl ring; and wherein D is not fused with D′.

In some embodiments, Ring D is phenyl. In one embodiment, Ring D is phenyl where the phenyl is independently substituted with one or two substituents selected from -halo and —N(R⁷)CO₂(C₁₋₆ aliphatic). In another embodiment, Ring D is phenyl where the phenyl is independently substituted with —F and —NHCO₂(C₁₋₃ aliphatic). In yet another embodiment, Ring D is phenyl, where the phenyl is independently substituted with —F and —NHCO₂(cyclopropyl). In one embodiment, Ring D is

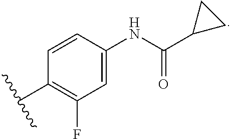

In other embodiments, Ring D is pyridinyl.

In some embodiments, R⁷ is fluoro. In other embodiments, R⁷ is OCH₃.

In some embodiments, T is absent.

In some embodiments, R^{Y} is —Z—R¹⁰.

In other embodiments, Z is absent. In some embodiments, Z is a C₁₋₆ alkylidene chain wherein 1-2 methylene units of Z is optionally replaced by O, —N(R⁴)—, or S. In other embodiments, Z is a C₁₋₄ alkylidene chain.

In another aspect of this invention, R¹⁰ is a 5-6 membered heterocyclic ring containing 1 nitrogen atom. In some embodiments, R¹⁰ is an optionally substituted pyrrolidine. In other embodiments, R¹⁰ is an optionally substituted piperidine. In some embodiments, said heterocyclic ring is attached to Z via a nitrogen atom.

In some embodiments, R¹⁰ is

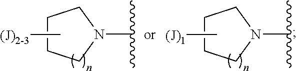

wherein
n is 1 or 2; and J is as defined herein.

In one embodiment, R^{Y} is

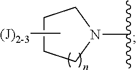

wherein n is 1 or 2. In some embodiments, each J is independently C₁₋₆alkyl, F, —N(R⁴)₂, CN, or —OR; or two J groups, together with the atom(s) to which they are bound, form a 4-7 membered heterocyclyl ring containing 1-2 heteroatoms selected from N or O; wherein said ring is optionally substituted with 0-3 J^{R}.

In some embodiments, at least one R⁴ of each —N(R⁴)₂ group is not H.

In other embodiments R is H, C₁₋₄alkyl or C₃₋₆ cycloalkyl; wherein said C₁₋₄alkyl or C₃₋₆ cycloalkyl is optionally substituted with 1-3 fluorine atoms.

In yet other embodiments R⁴ is H, C₁₋₅alkyl, or C₃₋₆ cycloalkyl; or two R⁴, together with the nitrogen atom to which they are bound, form a 3-6 membered monocyclic ring containing 1-2 heteroatoms selected from O, N, or S; wherein said monocyclic ring is optionally substituted with 0-3 J^{R}.

In some embodiments, at least one R⁴ of each —N(R⁴)₂ group is not H. In some embodiments, J^{R} is halo, C₁₋₃alkyl, or —O(C₁₋₃alkyl).

In another embodiment, R^{Y} is

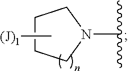

wherein n is 1 or 2. In some embodiments, J is F, —N(R⁴)₂, CN, —OR, oxo (═O), or C₂₋₆alkyl optionally substituted with 1 occurrence of OH or OCH₃. In some embodiments, at least one R⁴ of each —N(R⁴)₂ group is not H. In some embodiments, J is F.

In one embodiment,
Z is absent;
R^{Y} is

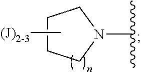

n is 2; and
each J is independently C₁₋₆alkyl, F, —N(R⁴)₂, CN, or —OR.

In some embodiments, at least one R⁴ of each —N(R⁴)₂ group is not H.

In another embodiment,
Z is absent;
R^{Y} is

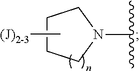

n is 2; and
two J groups, together with the atom(s) to which they are bound, form a 4-7 membered heterocyclyl ring containing 1-2 heteroatoms selected from N or O.

In some embodiments, said heterocyclyl ring is a 4-7 membered spirocyclic heterocyclyl ring containing 1-2 heteroatoms selected from N or O. In some embodiments, said spirocyclic heterocyclyl is a 5-membered spirocyclic heterocyclyl ring containing 1 heteroatom selected from N or O. In some embodiments, said 5-membered spirocyclic heterocyclyl ring contains 1 N (nitrogen) heteroatom. In some embodiments, said ring formed by the two J groups is optionally substituted with 0-3 J^{R}. In some embodiments, said ring formed by the two J groups is optionally substituted with 1 J^{R}.

In some embodiments, R^{Y} is

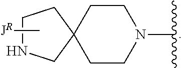

In other embodiments, R^{Y} is

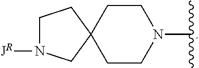

In some embodiments, J^{R} is CH₃.

Another aspect of this invention provides compounds wherein
R^{Y} is

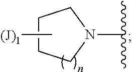

n is 1;
J is F, —N(R⁴)₂, CN, —OR, oxo (═O), or C₂₋₆alkyl optionally substituted with 1 occurrence of OH or OCH₃; and R¹ is substituted with 1 occurrence of —NHC(O)(C₁₋₆aliphatic) wherein said C₁₋₆aliphatic is substituted with 0-6 halo.

In some embodiments, at least one R⁴ of each —N(R⁴)₂ group is not H.

Another aspect of this invention provides compounds wherein
R^{Y} is

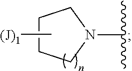

n is 1;
J is F; and
R¹ is substituted with 1 occurrence of —NHC(O) (C₁₋₆aliphatic) wherein said C₁₋₆aliphatic is substituted with 0-6 halo.

In some embodiments, R^{Y} is

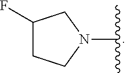

In other embodiments, R^{Y} is

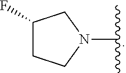

For purposes of this invention, the chemical elements are identified in accordance with the Periodic Table of the Elements, CAS version, Handbook of Chemistry and Physics, 75^{th} Ed. Additionally, general principles of organic chemistry are described in texts known to those of ordinary skill in the art, including, for example, “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As described herein, a specified number range of atoms includes any integer therein. For example, a group having from 1-4 atoms could have 1, 2, 3, or 4 atoms.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, and the like, as used herein, means an unbranched or branched, straight-chain or cyclic, substituted or unsubstituted hydrocarbon that is completely saturated or that contains one or more units of unsaturation that has a single point of attachment to the rest of the molecule. Suitable aliphatic groups include, but are not limited to, linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl.

The term “cycloaliphatic” (or “carbocycle” or “carbocyclyl” or “cycloalkyl” and the like) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable cycloaliphatic groups include, but are not limited to, cycloalkyl and cycloalkenyl groups. Specific examples include, but are not limited to, cyclohexyl, cyclopropenyl, and cyclobutyl.

The term “alkyl” as used herein, means an unbranched or branched, straight-chain hydrocarbon that is completely saturated and has a single point of attachment to the rest of the molecule. Specific examples of alkyl groups include, but are not limited to, methyl, ethyl, isopropyl, n-propyl, and sec-butyl.

The term “cycloalkyl” refers to a monocyclic hydrocarbon that is completely saturated and has a single point of attachment to the rest of the molecule. Suitable cycloalkyl groups include, but are not limited to, cyclopropyl, cyclobutyl, and cyclopentyl.

In the compounds of this invention, rings include linearly-fused, bridged, or spirocyclic rings. Examples of bridged cycloaliphatic groups include, but are not limited to, bicyclo[3.3.2] decane, bicyclo[3.1.1] heptane, and bicyclo[3.2.2] nonane.

The term “heterocycle”, “heterocyclyl”, or “heterocyclic”, and the like, as used herein means non-aromatic, monocyclic or bicyclic ring in which one or more ring members are an independently selected heteroatom. In some embodiments, the “heterocycle”, “heterocyclyl”, or “heterocyclic” group has three to ten ring members in which one or more ring members is a heteroatom independently selected from oxygen, sulfur, nitrogen, or phosphorus, and each ring in the system contains 3 to 7 ring members. Examples of bridged heterocycles include, but are not limited to, 7-aza-bicyclo[2.2.1]heptane and 3-aza-bicyclo[3.2.2]nonane.

Suitable heterocycles include, but are not limited to, 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

As used herein, the term “Ht” is interchangeable with “Het” and

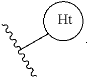

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “aryl” refers to monocyclic, or bicyclic ring having a total of five to twelve ring members, wherein at least one ring in the system is aromatic and wherein each ring in the system contains 3 to 7 ring members. The term “aryl” may be used interchangeably with the term “aryl ring”. The term “aryl” also refers to heteroaryl ring systems as defined hereinbelow.

The term “heteroaryl”, refers to monocyclic or bicyclic ring having a total of five to twelve ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”. Suitable heteroaryl rings include, but are not limited to, 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “unsaturated”, as used herein, means that a moiety has one or more units of unsaturation.

The term “halogen” means F, Cl, Br, or I.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W., Wuts, P. G in “Protective Groups in Organic Synthesis”, Third Edition, John Wiley & Sons, New York: 1999, and other editions of this book, the entire contents of which are hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

Unless otherwise indicated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise indicated, all tautomeric forms of the compounds of the invention are within the scope of the invention. As would be understood by a skilled practitioner, a pyrazole group can be represented in a variety of ways. For example, a structure drawn as

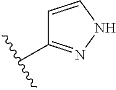

also represents other possible tautomers, such as

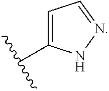

Likewise, a structure
drawn as

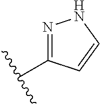

also represents other possible tautomers, such as

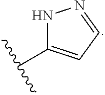

Unless otherwise indicated, a substituent can freely rotate around any rotatable bonds. For example, a substituent drawn as

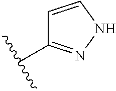

also represents

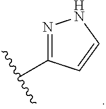

Likewise, a substituent drawn as

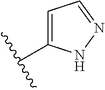

also represents

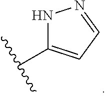

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C— enriched carbon are within the scope of this invention. Such compounds are useful, for example, as analytical tools or probes in biological assays.

The compounds of this invention may be prepared in light of the specification using steps generally known to those of ordinary skill in the art. Those compounds may be analyzed by known methods, including but not limited to LCMS (liquid chromatography mass spectrometry) and NMR (nuclear magnetic resonance). It should be understood that the specific conditions shown below are only examples, and are not meant to limit the scope of the conditions that can be used for making compounds of this invention. Instead, this invention also includes conditions that would be apparent to those skilled in that art in light of this specification for making the compounds of this invention. Unless otherwise indicated, all variables in the following schemes are as defined herein.

The following abbreviations are used:

HPLC is high performance liquid chromatography
LCMS liquid chromatography mass spectrometry
¹H NMR is nuclear magnetic resonance

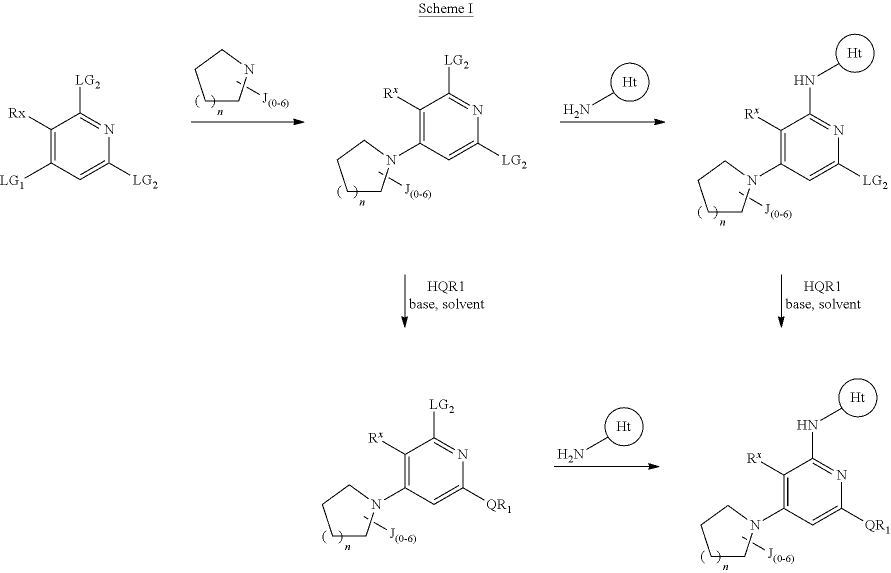

Scheme I above shows a generic method for making compounds of this invention wherein X¹ is N, X² is CH, and X³ is CR^{X}. In the above scheme, LG₁ is Cl or NO₂; LG₂ is Cl or Br.

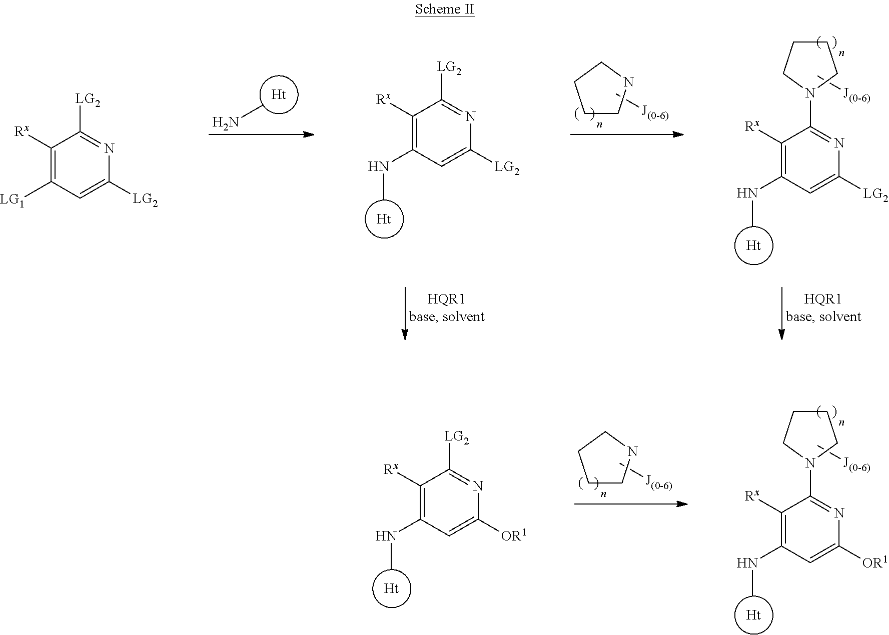

Scheme II above shows a generic method for making compounds of this invention wherein X¹ is CH, X² is N, and X³ is CR^{X}. In the above scheme, LG₁ is Cl or NO₂; LG₂ is Cl or Br.

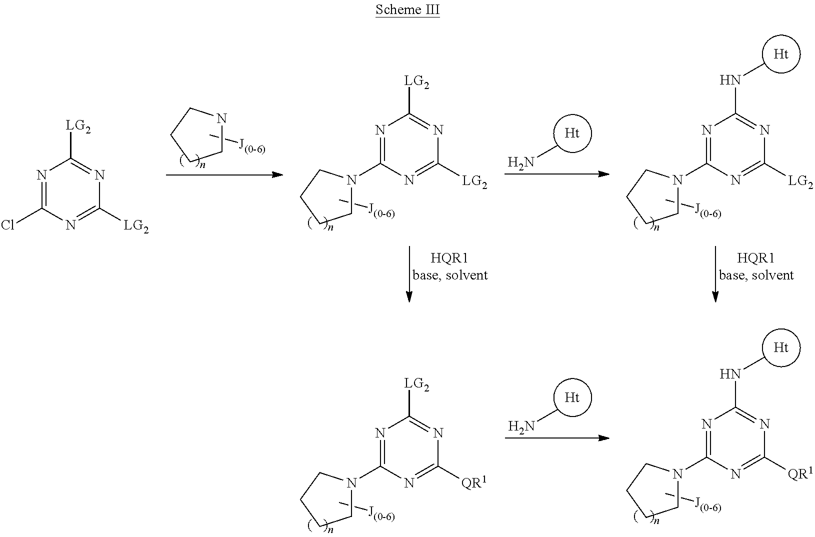

Scheme III above shows a generic method for making compounds of this invention wherein X¹, X², and X³ are N.

There are three main groups that are added to the triazine starting material. The order in which these groups are added can vary. The three main reactions involved are: addition of the pyrrolidine or piperidine, addition of the amino-heteroaryl, and addition of -Q-R¹. The pyrrolidine or piperidine, amino-heteroaryl, and -Q-R¹ can be added in various different orders. For instance, the amino-heteoraryl can be added first, followed by addition of the pyrrolidine or piperidine and finally addition of -Q-R¹. Or instead, addition of -Q-R¹ can occur first, followed by addition of the amino-heteroaryl, and finally addition of the pyrrolidine or piperidine. A skilled practitioner would understand the various reactions shown above.

In the above scheme, LG₂ is Cl or Br.

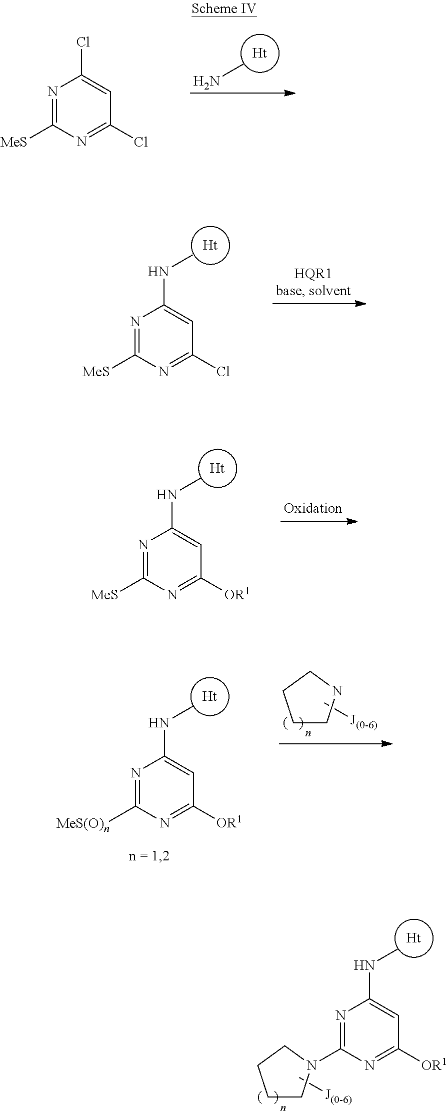

Scheme IV above shows a generic method for making compounds of this invention wherein X¹ is CH, X² is N, and X³ is N.

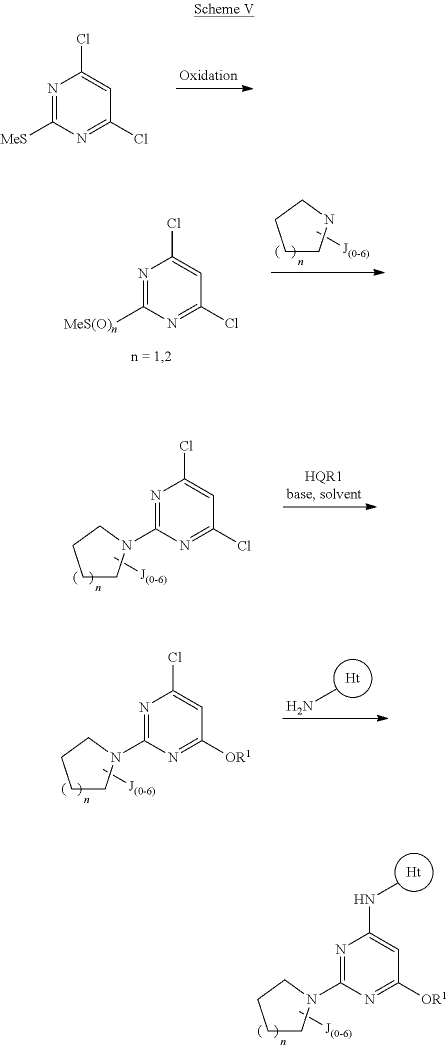

Scheme V above shows another generic method for making compounds of this invention wherein X¹ is CH, X² is N, and X³ is N. In Scheme V above, the order of the last two steps can be reversed. For example, the amino-heteroaryl can be added before HQ-R¹ is added.

Additionally, the compounds of this invention may be prepared according to the methods shown in WO2002/057259, the contents of which are incorporated by reference.

Accordingly, this invention relates to processes for making the compounds of this invention.

Methods for evaluating the activity of the compounds of this invention (e.g., kinase assays) are known in the art and are also described in the examples set forth.

The activity of the compounds as protein kinase inhibitors may be assayed in vitro, in vivo or in a cell line. In vitro assays include assays that determine inhibition of either the kinase activity or ATPase activity of the activated kinase. Alternate in vitro assays quantitate the ability of the inhibitor to bind to the protein kinase and may be measured either by radiolabelling the inhibitor prior to binding, isolating the inhibitor/kinase complex and determining the amount of radiolabel bound, or by running a competition experiment where new inhibitors are incubated with the kinase bound to known radioligands.

Another aspect of the invention relates to inhibiting kinase activity in a biological sample, which method comprises contacting said biological sample with a compound of formula I or a composition comprising said compound. The term “biological sample”, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof.

Inhibition of kinase activity in a biological sample is useful for a variety of purposes that are known to one of skill in the art. Examples of such purposes include, but are not limited to, blood transfusion, organ-transplantation, biological specimen storage, and biological assays.

Inhibition of kinase activity in a biological sample is also useful for the study of kinases in biological and pathological phenomena; the study of intracellular signal transduction pathways mediated by such kinases; and the comparative evaluation of new kinase inhibitors.

The Aurora protein kinase inhibitors or pharmaceutical salts thereof may be formulated into pharmaceutical compositions for administration to animals or humans. These pharmaceutical compositions, which comprise an amount of the Aurora protein inhibitor effective to treat or prevent an Aurora-mediated condition and a pharmaceutically acceptable carrier, are another embodiment of the present invention.

The term “Aurora-mediated condition” or “Aurora-mediated disease” as used herein means any disease or other deleterious condition in which Aurora (Aurora A, Aurora B, and Aurora C) is known to play a role. Such conditions include, without limitation, cancer, proliferative disorders, and myeloproliferative disorders.

Examples of myeloproliferative disorders include, but are not limited, to, polycythemia vera, thrombocythemia, myeloid metaplasia with myelofibrosis, chronic myelogenous leukaemia (CML), chronic myelomonocytic leukemia, hypereosinophilic syndrome, juvenile myelomonocytic leukemia, and systemic mast cell disease.

The term “cancer” also includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal; rectum, Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma] hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions. In some embodiments, the cancer is selected from colorectal, thyroid, or breast cancer.

In some embodiments, the compounds of this invention are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease.

In some embodiments, the compounds of this invention are useful for treating hematopoietic disorders, in particular, acute-myelogenous leukemia (AML), chronic-myelogenous leukemia (CML), acute-promyelocytic leukemia (APL), and acute lymphocytic leukemia (ALL).

In addition to the compounds of this invention, pharmaceutically acceptable derivatives or prodrugs of the compounds of this invention may also be employed in compositions to treat or prevent the above-identified disorders.

A “pharmaceutically acceptable derivative or prodrug” means any pharmaceutically acceptable ester, salt of an ester or other derivative of a compound of this invention which, upon administration to a recipient, is capable of providing, either directly or indirectly, a compound of this invention or an inhibitorily active metabolite or residue thereof. Such derivatives or prodrugs include those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Examples of pharmaceutically acceptable prodrugs of the compounds of this invention include, without limitation, esters, amino acid esters, phosphate esters, metal salts and sulfonate esters.

The compounds of this invention can exist in free form for treatment, or where appropriate, as a pharmaceutically acceptable salt.

As used herein, the term “pharmaceutically acceptable salt” refers to salts of a compound which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Base addition salts can be prepared by 1) reacting the purified compound in its acid form with a suitable organic or inorganic base and 2) isolating the salt thus formed.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

Base addition salts also include alkali or alkaline earth metal salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intraperitoneal, intrahepatic, intralesional and intracranial injection or infusion techniques.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, a bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used may include lactose and corn starch. Lubricating agents, such as magnesium stearate, may also be added. For oral administration in a capsule form, useful diluents may include lactose and dried cornstarch. When aqueous suspensions are required for oral use, the active ingredient may be combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials may include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations may be prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention may include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers may include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions may be prepared as solutions in saline, employing benzyl alcohol or other suitable preservatives, absorption promoters to enhance bioavailability, fluorocarbons, and/or other conventional solubilizing or dispersing agents.

The amount of kinase inhibitor that may be combined with the carrier materials to produce a single dosage form will vary depending upon the host treated, the particular mode of administration, and the indication. In an embodiment, the compositions should be formulated so that a dosage of between 0.01-100 mg/kg body weight/day of the inhibitor can be administered to a patient receiving these compositions. In another embodiment, the compositions should be formulated so that a dosage of between 0.1-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of inhibitor will also depend upon the particular compound in the composition.

According to another embodiment, the invention provides methods for treating or preventing cancer, a proliferative disorder, or a myeloproliferative disorder comprising the step of administering to a patient one of the herein-described compounds or pharmaceutical compositions.

The term “patient”, as used herein, means an animal, including a human.

In some embodiments, said method is used to treat or prevent a hematopoietic disorder, such as acute-myelogenous leukemia (AML), acute-promyelocytic leukemia (APL), chronic-myelogenous leukemia (CML), or acute lymphocytic leukemia (ALL).

In other embodiments, said method is used to treat or prevent myeloproliferative disorders, such as polycythemia vera, thrombocythemia, myeloid metaplasia with myelofibrosis, chronic myelogenous leukaemia (CML), chronic myelomonocytic leukemia, hypereosinophilic syndrome, juvenile myelomonocytic leukemia, and systemic mast cell disease.

In yet other embodiments, said method is used to treat or prevent cancer, such as cancers of the breast, colon, prostate, skin, pancreas, brain, genitourinary tract, lymphatic system, stomach, larynx and lung, including lung adenocarcinoma, small cell lung cancer, and non-small cell lung cancer.

Another embodiment provides a method of treating or preventing cancer comprising the step of administering to a patient a compound of formula I or a composition comprising said compound.

Another aspect of the invention relates to inhibiting kinase activity in a patient, which method comprises administering to the patient a compound of formula I or a composition comprising said compound. In some embodiments, said kinase is an Aurora kinase (Aurora A, Aurora B, Aurora C), Abl, Arg, FGFR1, MELK, MLK1, MuSK, Ret, or TrkA.

Depending upon the particular conditions to be treated or prevented, additional drugs may be administered together with the compounds of this invention. In some cases, these additional drugs are normally administered to treat or prevent the same condition. For example, chemotherapeutic agents or other anti-proliferative agents may be combined with the compounds of this invention to treat proliferative diseases.

Another aspect of this invention is directed towards a method of treating cancer in a subject in need thereof, comprising the sequential or co-administration of a compound of this invention or a pharmaceutically acceptable salt thereof, and another therapeutic agent. In some embodiments, said additional therapeutic agent is selected from an anti-cancer agent, an anti-proliferative agent, or a chemotherapeutic agent.

In some embodiments, said additional therapeutic agent is selected from camptothecin, the MEK inhibitor: U0126, a KSP (kinesin spindle protein) inhibitor, adriamycin, interferons, and platinum derivatives, such as Cisplatin.

In other embodiments, said additional therapeutic agent is selected from taxanes; inhibitors of bcr-abl (such as Gleevec, dasatinib, and nilotinib); inhibitors of EGFR (such as Tarceva and Iressa); DNA damaging agents (such as cisplatin, oxaliplatin, carboplatin, topoisomerase inhibitors, and anthracyclines); and antimetabolites (such as AraC and 5-FU).

In one embodiment, said additional therapeutic agent is dasatinib or nilotinib.

In one embodiment, said additional therapeutic agent is dasatinib.

In one embodiment, said additional therapeutic agent is nilotinib.

In yet other embodiments, said additional therapeutic agent is selected from camptothecin, doxorubicin, idarubicin, Cisplatin, taxol, taxotere, vincristine, tarceva, the MEK inhibitor, U0126, a KSP inhibitor, vorinostat, Gleevec, dasatinib, and nilotinib.

In another embodiment, said additional therapeutic agent is selected from Her-2 inhibitors (such as Herceptin); HDAC inhibitors (such as vorinostat), VEGFR inhibitors (such as Avastin), c-KIT and FLT-3 inhibitors (such as sunitinib), BRAF inhibitors (such as Bayer's BAY 43-9006) MEK inhibitors (such as Pfizer's PD0325901); and spindle poisons (such as Epothilones and paclitaxel protein-bound particles (such as Abraxane®).

Other therapies or anticancer agents that may be used in combination with the inventive anticancer agents of the present invention include surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, dexamethasone, and cyclophosphamide.

A compound of the instant invention may also be useful for treating cancer in combination with the following therapeutic agents: abarelix (Plenaxis Depot®; aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®; celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); zoledronate (Zometa®) and vorinostat (Zolinza®).

For a comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Another embodiment provides a simultaneous, separate or sequential use of a combined preparation.

Those additional agents may be administered separately, as part of a multiple dosage regimen, from the kinase inhibitor-containing compound or composition. Alternatively, those agents may be part of a single dosage form, mixed together with the kinase inhibitor in a single composition.

In order that this invention be more fully understood, the following preparative and testing examples are set forth. These examples are for the purpose of illustration only and are not to be construed as limiting the scope of the invention in any way. All documents cited herein are hereby incorporated by reference.

### EXAMPLES

As used herein, the term “Rt(min)” refers to the HPLC retention time, in minutes, associated with the compound. Unless otherwise indicated, the HPLC method utilized to obtain the reported retention time is as follows:
Column: ACE C8 column, 4.6×150 mm
Gradient: 0-100% acetonitrile+methanol 60:40 (20 mM Tris phosphate)
Flow rate: 1.5 mL/minute
Detection: 225 nm.

Mass spec. samples were analyzed on a MicroMass Quattro Micro mass spectrometer operated in single MS mode with electrospray ionization. Samples were introduced into the mass spectrometer using chromatography. Mobile phase for all mass spec. analyses consisted of 10 mM pH 7 ammonium acetate and a 1:1 acetonitrile-methanol mixture, column gradient conditions was 5%-100% acetonitrile-methanol over 3.5 mins gradient time and 5 mins run time on an ACE C8 3.0×75 mm column. Flow rate was 1.2 ml/min.

¹H-NMR spectra were recorded at 400 MHz using a Bruker DPX 400 instrument. The following compounds of formula I were prepared and analyzed as follows.

### Example 1

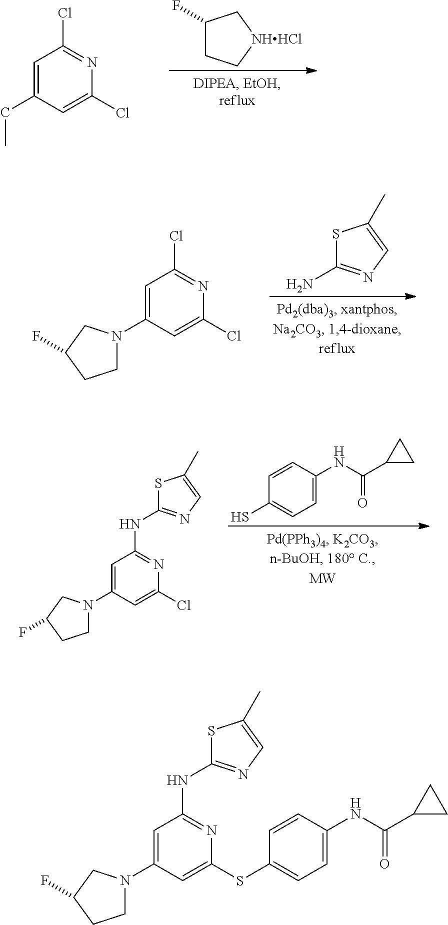

### (S)-N-(4-(4-(3-Fluoropyrrolidin-1-yl)-6-(5-methylthiazol-2-ylamino)pyridin-2-ylthio)phenyl)cyclopropanecarboxamide (Compound 1)

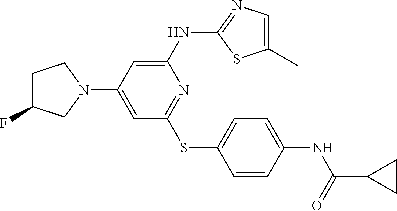

### (S)-2,6-Dichloro-4-(3-fluoropyrrolidin-1-yl)pyridine (4a)

To a mixture of (S)-(+)-3-fluoropyrrolidine hydrochloric acid (2.0 g, 15.9 mmol) and DiPEA (6.1 mL, 35 mmol) in ethanol (50 mL) was added 2,4,6-trichloropyridine (2.9 g, 15.9 mmol). The mixture was refluxed for one hour and then evaporated to dryness. The residue was purified by ISCO (EtOAc/heptanes; TLC: SiO2, EtOAc/heptanes=1:4, Rf=0.5 (2-substituted pyridine), Rf=0.2 (4-substituted pyridine) to yield 0.70 g (19%) of the desired product.

¹H-NMR (300 MHz, CDCl₃): δ 6.37 (s, 2H); 5.51-5.48 (m, ½H); 5.33-5.31 (m, ½H); 3.64-3.59 (m, 1H); 3.53-3.47 (m, 3H); 2.51-2.39 (m, 1H); 2.39-2.08 (m, 1H) ppm.

### (S)-6-Chloro-4-(3-fluoropyrrolidin-1-yl)-N-(5-methylthiazol-2-yl)pyridin-2-amine

Nitrogen was bubbled through a mixture of (S)-2,6-Dichloro-4-(3-fluoropyrrolidin-1-yl)pyridine (1.73 g, 7.36 mmol), 2-amino-5-methylthiazole (0.92 g, 8.1 mmol), Pd2 dba3 (0.34 g, 0.37 mmol), xantphos (0.32 g, 0.55 mmol), and Na2CO3 (1.1 g, 10.3 mmol) in dioxane. The mixture was heated in the microwave to 180° C. for one hour. HPLC analysis indicated complete conversion and the mixture was filtered over Celite. After a dioxane rinse of the Celite, the combined filtrates were evaporated to dryness. The residue was purified by column chromatography (SiO2 (100 mL), EtOAc/heptanes=1:9-1:0). Fractions with Rf=0.5-0.8 (TLC, SiO2, EtOAc) were pooled and evaporated to dryness to give 1.4 g of the desired product with a purity of 58-71% (HPLC, Rf=8.578 minutes).

¹H-NMR (300 MHz, CDCl₃): δ 7.00 (s, 1H); 6.11 (s, 1H); 5.99 (s, 1H); 5.47-5.44 (m, ½H); 5.29-5.27 (m, ½H); 3.61-3.47 (m, 4H); 2.52-2.40 (m, 1H); 2.38 (s, 3H); 2.28-2.04 (m, 1H) ppm.

### (S)-N-(4-(4-(3-Fluoropyrrolidin-1-yl)-6-(5-methylthiazol-2-ylamino)pyridin-2-ylthio)phenyl)cyclopropanecarboxamide

A mixture of (S)-6-Chloro-4-(3-fluoropyrrolidin-1-yl)-N-(5-methylthiazol-2-yl)pyridin-2-amine (0.5 g, 1.6 mmol), N-(4-mercaptophenyl)cyclopropanecarboxamide (330 mg, 1.7 mmol), potassium carbonate (500 mg, 3.6 mmol), and tetrakis(triphenylphosphine)palladium(0) (120 mg, 0.1 mmol) in 1-methyl-2-pyrrolidinone (NMP, 10 mL) was flushed with nitrogen for 15 minutes. The mixture was heated in the microwave to 180° C. for one hour. HPLC indicated complete conversion. The mixture was filtered over Celite. The Celite was rinsed with methanol. The combined filtrates were evaporated under reduced pressure to remove the methanol. Water (25 mL) was added to the residue under stirring. Stirring was continued for half an hour and the formed solids were collected by filtration and washed with water. The solids were coated on silica by dissolving it in methanol. The silica was brought on a column that was then eluted with a CH2Cl2/4-6% 2-propanol. Product containing fractions (TLC (SiO2 CH2Cl2/8% 2-propanol) Rf=0.65) were pooled and evaporated to dryness to yield 200 mg of a product with 66-72% purity (HPLC). This material was purified by preparative HPLC. After evaporation and lyophilization 41 mg (5.5%) of (S)-N-(4-(4-(3-Fluoropyrrolidin-1-yl)-6-(5-methylthiazol-2-ylamino)pyridin-2-ylthio)phenyl)cyclopropanecarboxamide was obtained with 99+% purity (HPLC, Rf=8.598 minutes).

¹H-NMR (300 MHz, DMSO-d₆): δ 10.49 (s, 1H); 10.39 (s, 1H); 7.70 (d, J=8.6 Hz, 2H); 7.48 (d, J=8.6 Hz, 2H); 6.86 (s, 1H); 5.89 (d, J=1.8 Hz, 1H); 5.85 (d, J=1.8 Hz, 1H); 5.50-5.32 (m, 1H); 3.51-3.20 (m, 4H); 2.26-2.08 (m, 2H); 2.14 (s, 3H); 1.91-1.78 (m, 1H); 0.82-0.80 (m, 4H) ppm.

### Example 2

### (S)-N-(4-(4-(3-Fluoropyrrolidin-1-yl)-6-(3-methyl-1H-pyrazol-5-ylamino)pyridin-2-ylthio)phenyl)cyclopropanecarboxamide (Compound 2)

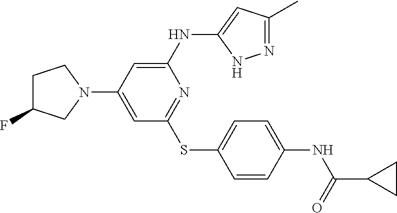

### (S)-6-Chloro-4-(3-fluoropyrrolidin-1-yl)-N-(3-methyl-1H-pyrazol-5-yl)pyridin-2-amine

Nitrogen was bubbled through a mixture of (S)-2,6-dichloro-4-(3-fluoropyrrolidin-1-yl)pyridine (600 mg, 2.6 mmol), tert-butyl 3-amino-5-methyl-1H-pyrazole-1-carboxylate (510 mg, 2.6 mmol), Pd2 dba3 (119 mg), xantphos (150 mg), and sodium carbonate (382 mg, 3.6 mmol) in dioxane (10 mL). The mixture was heated to 140° C. for 45 minutes and then to 170° C. for 15 minutes in the microwave. After filtration through Celite and a dioxane rinse, the solvents were removed under reduced pressure. The residue was purified by column chromatography (SiO2 (75 mL), CH2Cl2/2.5-7% 2-propanol; TLC: CH2Cl2/5% 2-propanol, Rf=0.3) to yield 230 mg of the desired product as an off-white solid (HPLC: Rf=7.341 minutes).

### (S)-N-(4-(4-(3-Fluoropyrrolidin-1-yl)-6-(3-methyl-1H-pyrazol-5-ylamino)pyridin-2-ylthio)phenyl)cyclopropanecarboxamide

After bubbling nitrogen through a mixture of (S)-6-chloro-4-(3-fluoropyrrolidin-1-yl)-N-(3-methyl-1H-pyrazol-5-yl)pyridin-2-amine (230 mg, 0.87 mmol), N-(4-mercaptophenyl)-cyclopropanecarboxamide (194 mg, 0.94 mmol), tetrakis(triphenylphosphine)-palladium (0) (90 mg), and K2CO3 (237 mg, 1.7 mmol) in NMP (5 mL) for 15 minutes, the mixture was heated to 190° C. for 1 hour. The mixture was poured in water (100 mL) and after half an hour of stirring the solids were collected by filtration and washed with water (twice). The solid was dried and purified by column chromatography (SiO2, CH2Cl2/2-10% 2-propanol). The fraction with Rf=0.1 (TLC: SiO2, CH2Cl2/5% 2-propanol) were pooled and evaporated to dryness to yield 300 mg with a purity of 80% (HPLC, Rf=8.08 minutes). This material was purified by preparative HPLC to give 45 mg of (S)-N-(4-(4-(3-Fluoropyrrolidin-1-yl)-6-(3-methyl-1H-pyrazol-5-ylamino)pyridin-2-ylthio)phenyl)cyclopropanecarboxamide after evaporation and lyophilization with a purity of 97+% (HPLC, Rf=8.092 minutes).

1H-NMR (300 MHz, DMSO-d6): 11.54 (s, 1H); 10.39 (s, 1H); 8.69 (s, 1H); 7.70 (d, J=8.5 Hz, 2H); 7.46 (d, J=8.6 Hz, 2H); 6.17 (s, 1H); 5.66-5.60 (m, 2H); 5.48-5.30 (m, 1H); 3.48-3.16 (m, 4H); 2.25-2.15 (m, 2H); 2.06 (s, 3H); 1.84-1.77 (m, 2H); 0.85-80 (m, 2H) ppm.

### Example 3

### Aurora-2 (Aurora A) Inhibition Assay

Compounds were screened for their ability to inhibit Aurora-2 using a standard coupled enzyme assay (Fox et al., Protein Sci., (1998) 7, 2249). Assays were carried out in a mixture of 100 mM Hepes (pH7.5), 10 mM MgCl₂, 1 mM DTT, 25 mM NaCl, 2.5 mM phosphoenolpyruvate, 300 μM NADH, 30 μg/ml pyruvate kinase and 10 μg/ml lactate dehydrogenase. Final substrate concentrations in the assay were 400 μM ATP (Sigma Chemicals) and 570 μM peptide (Kemptide, American Peptide, Sunnyvale, Calif.). Assays were carried out at 30° C. and in the presence of 40 nM Aurora-2.

An assay stock buffer solution was prepared containing all of the reagents listed above, with the exception of Aurora-2 and the test compound of interest. 55 μl of the stock solution was placed in a 96 well plate followed by addition of 2 μl of DMSO stock containing serial dilutions of the test compound (typically starting from a final concentration of 7.5 μM). The plate was preincubated for 10 minutes at 30° C. and the reaction initiated by addition of 10 μl of Aurora-2. Initial reaction rates were determined with a Molecular Devices SpectraMax Plus plate reader over a 10 minute time course. IC50 and Ki data were calculated from non-linear regression analysis using the Prism software package (GraphPad Prism version 3.0cx for Macintosh, GraphPad Software, San Diego Calif., USA). Compounds 1 and 2 were found to inhibit Aurora A at a Ki value of <0.1 μM, respectively.

### Example 4

### Aurora-1 (Aurora B) Inhibition Assay(Radiometric)

An assay buffer solution was prepared which consisted of 25 mM HEPES (pH 7.5), 10 mM MgCl₂, 0.1% BSA and 10% glycerol. A 22 nM Aurora-B solution, also containing 1.7 mM DTT and 1.5 mM Kemptide (LRRASLG), was prepared in assay buffer. To 22 μL of the Aurora-B solution, in a 96-well plate, was added 2 μl of a compound stock solution in DMSO and the mixture allowed to equilibrate for 10 minutes at 25° C. The enzyme reaction was initiated by the addition of 16 μl stock [γ-³³P]-ATP solution (˜20 nCi/μL) prepared in assay buffer, to a final assay concentration of 800 μM. The reaction was stopped after 3 hours by the addition of 16 μL 500 mM phosphoric acid and the levels of ³³P incorporation into the peptide substrate were determined by the following method.

A phosphocellulose 96-well plate (Millipore, Cat no. MAPHNOB50) was pre-treated with 100 μL of a 100 mM phosphoric acid prior to the addition of the enzyme reaction mixture (40 μL). The solution was left to soak on to the phosphocellulose membrane for 30 minutes and the plate subsequently washed four times with 200 μL of a 100 mM phosphoric acid. To each well of the dry plate was added 30 μL of Optiphase ‘SuperMix’ liquid scintillation cocktail (Perkin Elmer) prior to scintillation counting (1450 Microbeta Liquid Scintillation Counter, Wallac). Levels of non-enzyme catalyzed background radioactivity were determined by adding 16 μL of the 500 mM phosphoric acid to control wells, containing all assay components (which acts to denature the enzyme), prior to the addition of the [γ-³³P]-ATP solution. Levels of enzyme catalyzed ³³P incorporation were calculated by subtracting mean background counts from those measured at each inhibitor concentration. For each Ki determination 8 data points, typically covering the concentration range 0-10 μM compound, were obtained in duplicate (DMSO stocks were prepared from an initial compound stock of 10 mM with subsequent 1:2.5 serial dilutions). Ki values were calculated from initial rate data by non-linear regression using the Prism software package (Prism 3.0, Graphpad Software, San Diego, Calif.). Compounds 1 and 2 were found to inhibit Aurora B at a Ki value of <1.0 μM.

### Example 5

### Analysis of Cell Proliferation and Viability

Compounds were screened for their ability to inhibit cell proliferation and their effects on cell viability using Colo205 cells obtained from ECACC and using the assay shown below.

Colo205 cells were seeded in 96 well plates and serially diluted compound was added to the wells in duplicate. Control groups included untreated cells, the compound diluent (0.1% DMSO alone) and culture medium without cells. The cells were then incubated for 72 or 96 hrs at 37 C in an atmosphere of 5% CO2/95% humidity.

To measure proliferation, 3 h prior to the end of the experiment 0.5 μCi of 3H thymidine was added to each well. Cells were then harvested and the incorporated radioactivity counted on a Wallac microplate beta-counter. Cell viability was assessed using Promega CellTiter 96AQ to measure MTS conversion. Dose response curves were calculated using either Prism 3.0 (GraphPad) or SoftMax Pro 4.3.1 LS (Molecular Devices) software.

While we have described a number of embodiments of this invention, it is apparent that our basic examples may be altered to provide other embodiments that utilize or encompass the compounds, methods, and processes of this invention. Therefore, it will be appreciated that the scope of this invention is to be defined by the appended claims.

We claim:

1. A compound of formula I: 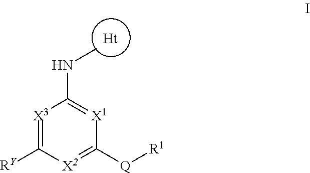
or a pharmaceutically acceptable salt thereof, wherein:
X¹ is N or CH;
X² is N or CH;
X³ is N or CR^{X};
provided that when X³ is CR^{X}, only one of X¹ and X² is N; and
provided that at least one of X¹, X² and X³ is N;
Ht is thiazole or pyrazole, wherein each ring is optionally and independently substituted with R² and R^{2′};
Q is —O—, —NR′—, —S—, —C(═O)—, or —C(R′)₂—;
R^{x} is H or F;
R^{Y} is —Z—R¹⁰;
R¹ is T-(Ring D);
Ring D is a 5-7 membered monocyclic aryl or heteroaryl ring, wherein said heteroaryl has 1-4 ring heteroatoms selected from O, N, or S; Ring D can optionally be fused with Ring D′;
Ring D′ is a 5-8 aromatic, partially saturated, or fully unsaturated ring containing 0-4 ring heteroatoms selected from nitrogen, oxygen, or sulfur;
Ring D and Ring D′ are each independently and optionally substituted with 0-4 occurrences of oxo or —W—R⁵;
each T is independently a C₁₋₄ alkylidene chain or is absent;
R² is H, C₁₋₃ alkyl, or cyclopropyl;
R^{2′} is H;
each Z and W is independently absent or a C₁₋₁₀ alkylidene chain wherein up to six methylene units of the alkylidene chain are optionally replaced by V;
each V is selected from —O—, —C(═O)—, —S(O)—, —S(O)₂—, —S—, or —N(R⁴)—;
each R⁵ is independently —R, -halo, —OR, —C(═O)R, —CO₂R, —COCOR, COCH₂COR, —NO₂, —CN, —S(O)R, —S(O)₂R, —SR, —N(R⁴)₂, —CON(R⁷)₂, —SO₂N(R⁷)₂, —OC(═O)R, —N(R⁷)COR, —N(R⁷)CO₂ (C₁₋₆ aliphatic), —N(R⁴)N(R⁴)₂, —C═NN(R⁴)₂, —C═N—OR, —N(R⁷)CON(R⁷)₂, —N(R⁷)SO₂N(R⁷)₂, —N(R⁴)SO₂R, or —OC(═O)N(R⁷)₂;
each R is H, a C₁₋₆ aliphatic group, a C₆₋₁₀ aryl ring, a heteroaryl ring having 5-10 ring atoms, or a heterocyclyl ring having 4-10 ring atoms; wherein said heteroaryl or heterocyclyl ring has 1-4 ring heteroatoms selected from nitrogen, oxygen, or sulfur; R is optionally substituted with 0-6 R⁹;
each R⁴ is —R⁷, —COR⁷, —CO₂R⁷, —CON(R⁷)₂, or —SO₂R⁷;
each R⁷ is independently H or C₁₋₆ aliphatic optionally substituted with 1-6 halo or —O(C₁₋₆alkyl); or two R⁷ on the same nitrogen are taken together with the nitrogen to form an optionally substituted 4-8 membered heterocyclyl or heteroaryl ring containing 1-4 heteroatoms selected from nitrogen, oxygen, or sulfur;
each R⁹ is —R′, -halo, —OR′, —C(═O)R′, —CO₂R′, —COCOR′, COCH₂COR′, —NO₂, —CN, —S(O)R′, —S(O)₂R′, —SR′, —N(R′)₂, —CON(R′)₂, —SO₂N(R′)₂, —OC(═O)R′, —N(R′)COR′, —N(R′)CO₂ (C₁₋₆ aliphatic), —N(R′)N(R′)₂, —N(R′)CON(R′)₂, —N(R′)SO₂N(R′)₂, —N(R′)SO₂R′, —OC(═O)N(R′)₂, ═NN(R′)₂, ═N—OR′, or ═O;
each R¹⁰ is a 5-6 membered heterocyclic ring containing 1 heteroatom selected from O, N, or S; each R¹⁰ is optionally substituted with 0-6 occurrences of J;
each J is independently R, -halo, —OR, oxo, —C(═O)R, —CO₂R, —COCOR, —COCH₂COR, —NO₂, —CN, —S(O)R, —S(O)₂R, —SR, —N(R⁴)₂, —CON(R⁷)₂, —SO₂N(R⁷)2, —OC(═O)R, —N(R⁷)COR, —N(R⁷)CO₂(C₁₋₆ aliphatic), —N(R⁴)N(R⁴)₂, ═NN(R⁴)₂, ═N—OR, —N(R⁷)CON(R⁷)₂, —N(R⁷)SO₂N(R⁷)₂, —N(R⁴)SO₂R, —OC(═O)N(R⁷)₂, or —OP(═O)(OR″)₂; or
2 J groups, on the same atom or on different atoms, together with the atom(s) to which they are bound, form a 3-8 membered saturated, partially saturated, or unsaturated ring having 0-2 heteroatoms selected from O, N, or S; wherein 1-4 hydrogen atoms on the ring formed by the 2 J groups is optionally replaced with J^{R}; or two hydrogen atoms on the ring are optionally replaced with oxo or a spiro-attached C₃₋₄ cycloalkyl; wherein said C₃₋₄ alkyl is optionally substituted with 1-3 fluorine;
each J^{R} is F or R^{7′};
each R^{7′} is independently C₁₋₆ aliphatic; —O(C₁₋₆ aliphatic); or a 5-6 membered heteroaryl containing 1-4 heteroatoms selected from O, N, or S; each R^{7′} is optionally substituted with 0-3 J⁷;
J⁷ is independently NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄aliphatic), or haloC₁₋₄aliphatic;
each R′ is independently H or a C₁₋₆aliphatic group; or two R′, together with atom(s) to which they are bound, form a 3-6 membered carbocyclyl or a 3-6 membered heterocyclyl containing 0-1 heteroatoms selected from O, N, or S; and
each R″ is independently H or C₁₋₂alkyl.

2-7. (canceled)

8. The compound of claim 1 selected from a compound of formula I-b, I-c, or I-f:

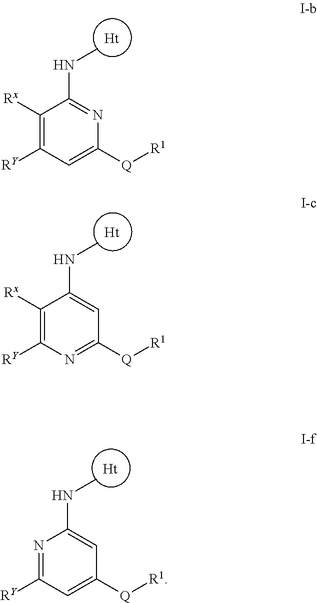

9. The compound of claim 8 selected from a compound of formula I-b.

10. The compound of claim 1, wherein Ht is

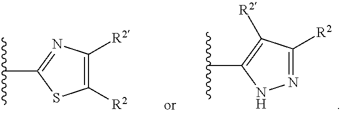

11. The compound of claim 1, wherein Q is —S—.

12. The compound of claim 1, wherein Q is —O—.

13. The compound of claim 1, wherein R² is H or C₁₋₃ alkyl.

14-24. (canceled)

25. The compound of claim 1, wherein Ring D is phenyl or pyridyl.

26. The compound of claim 25, wherein Ring D is phenyl.

27. The compound of claim 25, wherein Ring D is phenyl, wherein the phenyl is independently substituted with one or two substituents selected from -halo and —N(R⁷)CO₂(C₁₋₆ aliphatic).

28. The compound of claim 25, wherein Ring D is phenyl, wherein the phenyl is independently substituted with —F and —NHCO₂ (C₁₋₃ aliphatic).

29. The compound of claim 25, wherein Ring D is phenyl, wherein the phenyl is independently substituted with —F and —NHCO₂(cyclopropyl).

30. The compound of claim 25, wherein Ring D is

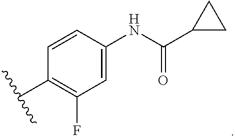

31. (canceled)

32. The compound of claim 30, wherein Z is absent.

33. The compound of claim 30, wherein Z is a C₁₋₆ alkylidene chain wherein 1-2 methylene units of Z is optionally replaced by O, —N(R⁴)—, or S.

34-41. (canceled)

42. The compound of claim 1, wherein R^{Y} is 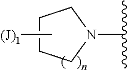
wherein n is 1 or 2.

43. The compound of claim 41, wherein J is F, —N(R⁴)₂, CN, —OR, oxo (═O), or C₂₋₆ alkyl optionally substituted with 1 occurrence of OH or OCH₃; wherein at least one R⁴ of each —N(R⁴)₂ group is not H.

44. The compound of claim 42, wherein J is F.

45. The compound claim 1, wherein n is 1.

46. The compound of claim 1, wherein n is 2.

47-56. (canceled)

57. The compound of claim 1, wherein
R^{Y} is 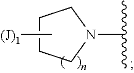
n is 1;
J is F, —N(R⁴)₂, CN, —OR, oxo (═O), or C₂₋₆alkyl optionally substituted with 1 occurrence of OH or OCH₃; wherein at least one R⁴ of each —N(R⁴)₂ group is not H;
R¹ is substituted with 1 occurrence of —NHC(O) (C₁₋₆aliphatic) wherein said C₁₋₆aliphatic is substituted with 0-6 halo.

58. The compound of claim 1, wherein
R^{Y} is 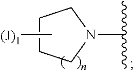
n is 1;
J is F; and
R¹ is substituted with 1 occurrence of —NHC(O) (C₁₋₆aliphatic) wherein said C₁₋₆aliphatic is substituted with 0-6 halo.

59. The compound according to claim 56, wherein R^{Y} is

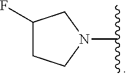

60. The compound according to claim 58, wherein R^{Y} is

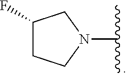

61. A composition comprising a compound of formula I: 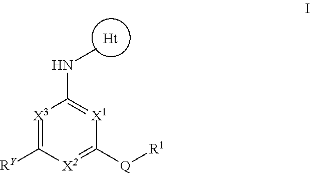
or a pharmaceutically acceptable salt thereof, and a pharmaceutically acceptable carrier, wherein the variables are defined according to claim 1.

62-72. (canceled)

73. The compound of claim 1 selected from the following:
(S)-N-(4-(4-(3-Fluoropyrrolidin-1-yl)-6-(5-methylthiazol-2-ylamino)pyridin-2-ylthio)phenyl)cyclopropanecarboxamide; and
(S)-N-(4-(4-(3-Fluoropyrrolidin-1-yl)-6-(3-methyl-1H-pyrazol-5-ylamino)pyridin-2-ylthio)phenyl)cyclopropanecarboxamide.
